(19) United States
(12) Patent Application Publication
(10) Pub. No.: US 2008/0009733 A1
(43) Pub. Date: Jan. 10, 2008

(54) Method for Evaluating Regional Ventricular Function and Incoordinate Ventricular Contraction

(75) Inventor: Sanjeev Saksena, Green Brook, NJ (US)

Correspondence Address: HANSEN HUANG TECHNOLOGY LAW GROUP, LLP, 1725 EYE STREET, NW, SUITE 300, WASHINGTON, DC, 20006, US

(73) Assignee: EP MEDSYSTEMS, INC., West Berlin, NJ (US)

(21) Appl. No.: 11/426,685

(22) Filed: Jun. 27, 2006

(51) Int. Cl. A61B 8/00

(52) U.S. Cl. 600/443

(57) ABSTRACT

A method for assessing cardiac function using an ultrasound imaging catheter system includes positioning an ultrasound catheter so the ultrasound transducer can image a ventricle, obtaining images of the ventricle at two or more times within the cardiac cycle, recognizing an edge of the endocardium, measuring dimensions of the ventricle, calculating a volume or area of the ventricle at the two or more points in the cardiac cycle, and calculating the ejection fraction based upon the difference in volume or area at the two or more times in the cardiac cycle. The method can be used to determine a location for an intervention, such as placement of a pacemaker pacing lead, and may be performed before and after an intervention to assess the impact of the treatment on cardiac function.

### FIELD OF THE INVENTION

This invention relates to medical diagnostic methods, and more particularly to methods for evaluating ventricular function and incoordinated ventricular contraction using intracardiac echocardiography.

### BACKGROUND OF THE INVENTION

Evaluating the ejection function and ejection characteristics of the heart is important to the proper diagnosis of cardiovascular health and selecting optimum therapies for treating cardiac disorders. A common measure of the ejection function is ejection fraction, which is the fraction of blood taken into the ventricle that is ejected in each contraction cycle. The higher the ejection fraction, the more of the blood volume is ejected from the ventricle in each beat. Current methods for evaluating heart function and ejection characteristics involve using an echocardiogram or cardiac blood pool scan test. During an echocardiogram a transducer is placed on the patient's ribs near the breast bone and directed toward the heart. The transducer picks up the echoes of sound waves and transmits them as electrical impulses. The echocardiography machine converts these impulses into moving pictures of the heart. The pitfall of the echocardiogram is that it is prone to noise and patient's lungs, ribs, or body tissue may prevent the sound waves and echoes from providing a clear picture of heart function sufficient to assess atrial function.

During a cardiac blood pool scan test, a small amount of radioactive tracer is injected into the patient's vein. A gamma camera is used to detect the radioactive tracer as it flows through the heart and lungs, and thereby measure the ejection fraction by calculating percentage of blood pumping out of the heart with each heartbeat. Downsides of the cardiac blood pool scan test include the potential allergic reactions to the radioactive tracer and the risks associated with exposure to radiation. Neither test is suitable for performance during another procedure such as an implant of a device such as a defibrillator or a pacemaker.

Currently no percutaneous method exists for evaluating the function and ejection characteristics of the heart. In addition, no percutaneous method currently exists in which regional ejection fraction during the systolic period can be monitored in a phased analysis of the regional wall motion to give a temporal sequence of regional ventricular ejection.

### SUMMARY OF THE INVENTION

The embodiments of the invention disclosed herein detail new methods for evaluating heart function and performance using the characteristics of ultrasound energy delivered via a phased array ultrasound imaging catheter positioned inside the heart.

The various embodiment methods establish quantitative evaluations of the pumping function in the heart muscle for ejection of blood from the heart to the great vessels supplying the body. The embodiment methods permit assessing the performance of individual regions of the lower chambers of the heart (i.e., the left and right ventricles) so that the overall pumping performance as well as the regional performance of each individual chamber of the heart can be assessed. Further, the embodiment methods monitor the regional ejection fraction during the systolic period in a phased analysis of the regional wall motion to give a temporal sequence of regional ventricular ejection. This phase analysis determines the timing of wall motion of these segments. An axial assessment can be performed along the long axis of the ventricle to determine apical to basal sequencing of ventricular ejection.

The various embodiment methods include generating a visual display for effectively communicating the measurements of heart function to a clinician. The quantitative evaluation results may be presented numerically superimposed over an image or stylized model of the heart or ventricle. Alternatively, the quantitative evaluation results may be presented using graphical means, including color coding to visually indicate ejection fraction and/or relative contraction lag or both on a global or regional basis. By providing a graphical display of the measured heart function superimposed over an image or stylized model of the heart, the various embodiments aid the clinician in identifying suitable or desirable locations for attaching pacing leads to the heart.

The various embodiment methods may be performed before and after an intervention in order to assess the impact of the procedure or therapy upon heart function. In an embodiment, the methods are performed before and after cardiac pacemaker settings are adjusted in order to help the clinician optimize settings, such as the timing of pacing, in order to compensate for cardiac conditions including incoordinate contraction.

The various embodiment methods may be used to help detect any pathology of the heart which causes changes in ejection characteristics of the ventricles. Such pathologies include but are not limited to: ischemia; infarction; mitral valve prolapse, stenosis or insufficiency; aortic valve stenosis or insufficiency; cardiac malformation; dilated, restrictive or hypertrophies cardiomyopathy; hydropericardium; and hemopericardium.

The various embodiments provide methods for evaluating the various chambers of the heart muscle through a percutaneous method to minimize the impact of the procedure on the patient while permitting a comprehensive review and evaluation of heart function.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention.

FIGS. 1A and 1B diagram steps for positioning an intracardiac phased array ultrasound transducer positioned within the right atrium for examining the right ventricle of a (human) heart.

FIGS. 2A and 2B diagram steps for positioning an intracardiac phased array ultrasound transducer positioned within the right ventricle for examining the left ventricle of a (human) heart.

FIG. 3 is a diagram of an alternative method for positioning an intracardiac phased array ultrasound transducer positioned within the right ventricle for examining the left ventricle of a (human) heart.

FIG. 4 is a functional system diagram of an ultrasound imaging system suitable for use in various embodiments.

FIG. 5 is a component system diagram of an ultrasound imaging system suitable for use in various embodiments.

FIG. 6 is a representation of a B-mode image of the left ventricle at diastole obtained by an intracardiac phased array ultrasound transducer positioned within the right ventricle.

FIG. 7 is a representation of a B-mode image of the left ventricle at systole obtained by an intracardiac phased array ultrasound transducer positioned within the right ventricle.

FIG. 8 is a representation of the left ventricle illustrating axes of measurement according to an embodiment.

FIG. 9 is a representation of the right ventricle illustrating axes of measurement according to an embodiment.

FIG. 10 is a representation of a ventricle including axes of measurements according to an embodiment.

FIG. 11 is a representation of a B-mode ultrasound image of the left ventricle at diastole with axes of measurement superimposed according to an embodiment.

FIG. 12 is a flowchart of the steps of an embodiment of the present invention.

### DETAILED DESCRIPTION OF THE PREFERRED EMBODIMENTS

The various embodiments will be described in detail with reference to the accompanying drawings. Wherever possible, the same reference numbers will be used throughout the drawings to refer to the same or like parts.

As used herein, the terms “about” or “approximately” for any numerical values or ranges indicates a suitable dimensional tolerance that allows the part or collection of components to function for its intended purpose as described herein. Also, as used herein, the terms “patient”, “host” and “subject” refer to any human or animal subject and are not intended to limit the systems or methods to human use, although use of the subject invention in a human patient represents a preferred embodiment.

The methods of the various embodiments enable physicians to obtain more complete and comprehensive visualizations of the function and status of the ventricles of the heart. In the various embodiments, ultrasonic imaging of the chambers of the heart via intracardiac echocardiography provide measurements to establish a quantitative evaluation of the pumping function of the heart as a whole and of the individual performance of the various heart chambers. The embodiment methods monitor the regional ejection fraction during the systolic period in a phased analysis of the regional wall motion to give a temporal sequence of regional ventricular ejection. This phase analysis determines the timing of wall motion of these segments. An axial assessment can be performed along the long axis of the ventricle to determine apical to basal sequencing of ventricular ejection. A user-friendly display of the analysis results is provided to aid the clinician in understanding the results. Presenting the analysis results superimposed upon an image or stylized model of the heart enables the clinician to recognize ventricle regions requiring intervention, such as pacing, and to position pacing leads, for example, for best therapeutic results. The various methods may also be performed before and after interventions to measure the impact of the therapies upon heart function. In an embodiment, the assessment methods are performed after cardiac pacemaker settings are adjusted to enable the clinician to identify settings, such as the timing of pacing of each lead, which optimize heart function.

The various embodiments involve placement of a phased array ultrasound imaging catheter within the various chambers of the heart. By imaging the various anatomies of the heart during the heart's normal operation, assessments of the hearts ability to eject the appropriate volume of blood can be made. Examples of phased array ultrasound imaging catheters suitable for placement in the left pulmonary artery for ventricular mapping and methods of using such devices in cardiac diagnosis are disclosed in U.S. Patent Application Publication Nos. 2004/0127798 to Dala-Krishna, et al., 2005/0228290 to Borovsky, et al., and 2005/0245822 to Dala-Krishna, et al., each of which is incorporated herein by reference in their entirety. A suitable phased array ultrasound imaging catheter is the ViewMate™ which is commercially available from EP MedSystems, Inc. of West Berlin, N.J.

An embodiment of the present invention provides a method to outline the inner lining of the ventricle during different phases of the cardiac cycle. The inner lying of the ventricle, the endocardium, can be imaged using ultrasound imaging techniques employing ultrasound delivered by a phased array ultrasound transducer mounted on a catheter, such as described in U.S. Patent Application Publication Nos. 2004/0127798 and 2005/0245822. Ultrasound energy is reflected from the endocardial surface and from tissue layers within the endocardium of the lower chamber of the heart. Reflected ultrasound is detected by the phased array ultrasound transducer, where the sound energy is converted into electrical pulses which can be processed to render a two-dimensional image of the inner lining of the heart. Ultrasound energy is also reflected from the heart valves and other anatomic structures allowing the ultrasound equipment to resolve the anatomic positions of these structures as well.

The methods of the various embodiments permit automated tracking as well as manual identification of the endocardial surface of the right and left ventricle.

To acquire images of the endocardial surface of the right and left ventricle, a phased array ultrasound imaging catheter is positioned within the heart via percutaneous cannulation using standard cardiac catheterization techniques of the femoral vein or the subclavian or jugular veins.

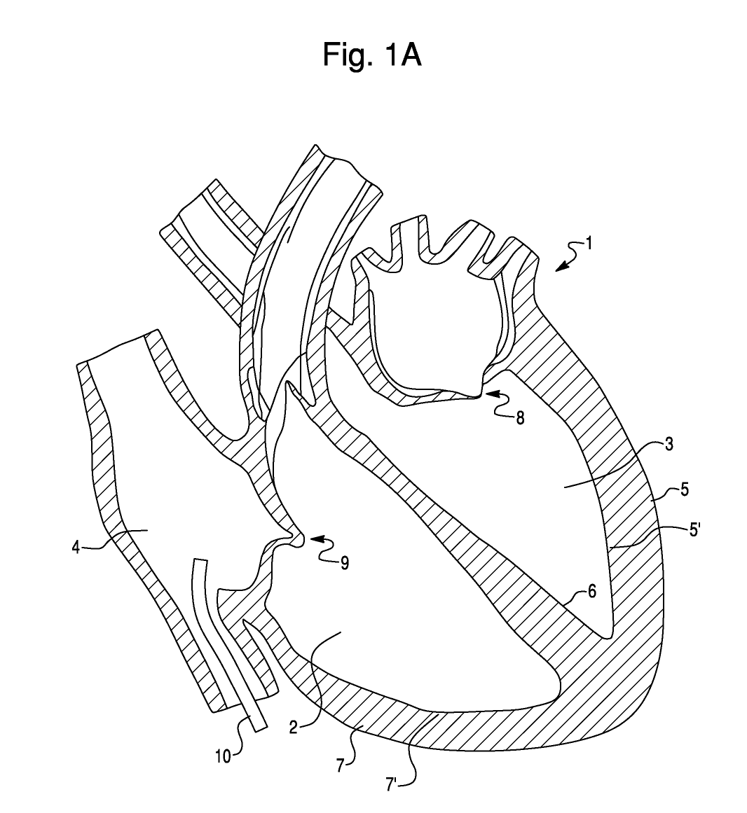

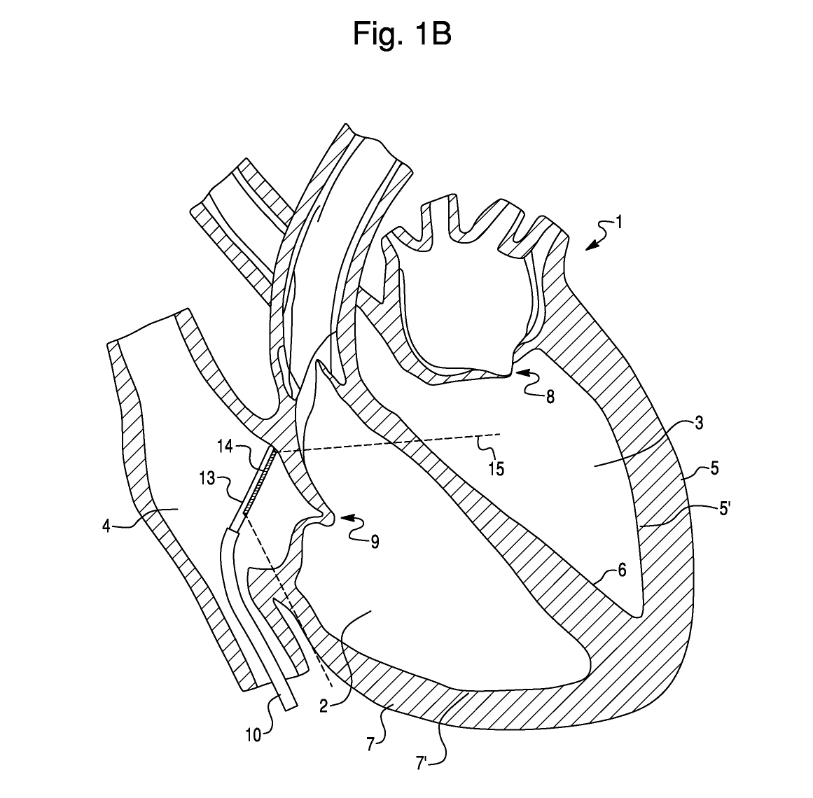

In order to properly position the phased array ultrasound imaging catheter, a long preformed intravascular sheath 10 is advanced under fluoroscopic control into the right atrium 4 of the heart 1, as shown in FIG. 1A. FIG. 1A illustrates access to the heart by cardiac catheterization via the femoral artery. A guide wire may be used to properly position the sheath in or near the orifice of the tricuspid valve. Once the sheath is positioned with its distal end in the right atrium 4, the phased array ultrasound imaging catheter 13 can be advanced through the sheath until the ultrasound transducer 14 is properly positioned outside the tricuspid valve 9 for imaging the right ventricle 2, as shown in FIG. 1B. In this position, the field of view 15 (indicated by dotted lines) of the ultrasound transducer 14 can address most, if not all of the right ventricle 2, right ventricle wall 7 and much of the septum 6.

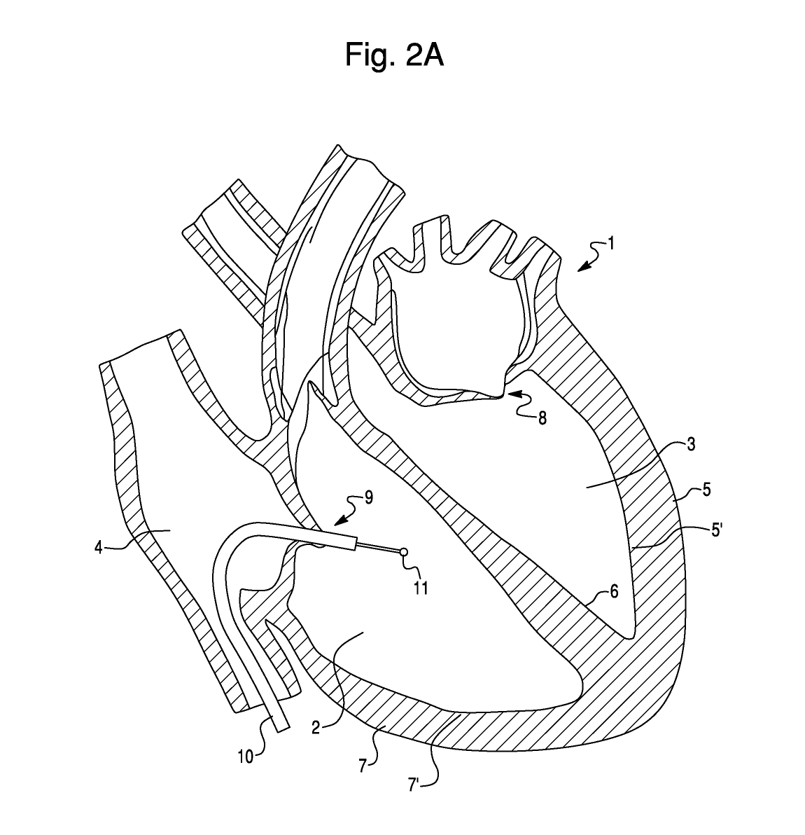

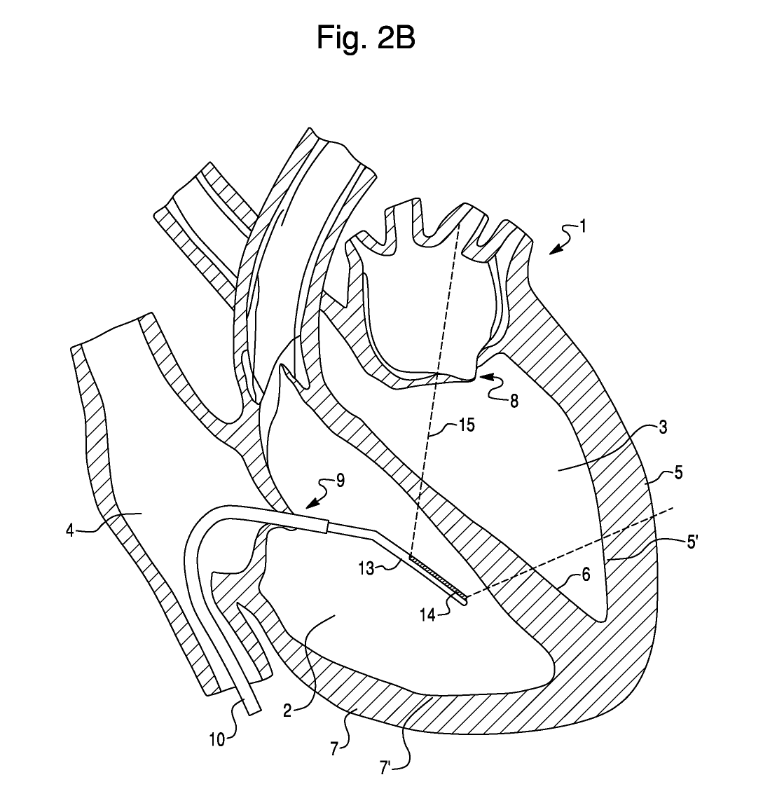

For imaging the left ventricle 3, the ultrasound transducer 14 needs to be positioned within the right ventricle. This can be accomplished by passing a guide wire 11 through the sheath 10, and under fluoroscopy control, passing the guide wire 11 through the tricuspid valve 9. The sheath 10 is then directed over the guide wire 11 into the orifice of the tricuspid valve 9 and advanced into the right ventricular cavity 2, as illustrated in FIG. 2A. Once the sheath 10 is properly positioned, the guide wire 11 is withdrawn and the phased array ultrasound imaging catheter 13 is advanced under fluoroscopic control through the sheath 10 into a position inside the right ventricular inflow tract in the mid cavity region, as illustrated in FIG. 2B. In this position, the field of view 15 (indicated by dotted lines) of the ultrasound transducer 14 can address most, if not all of the left ventricle wall 5 and much of the septum 6.

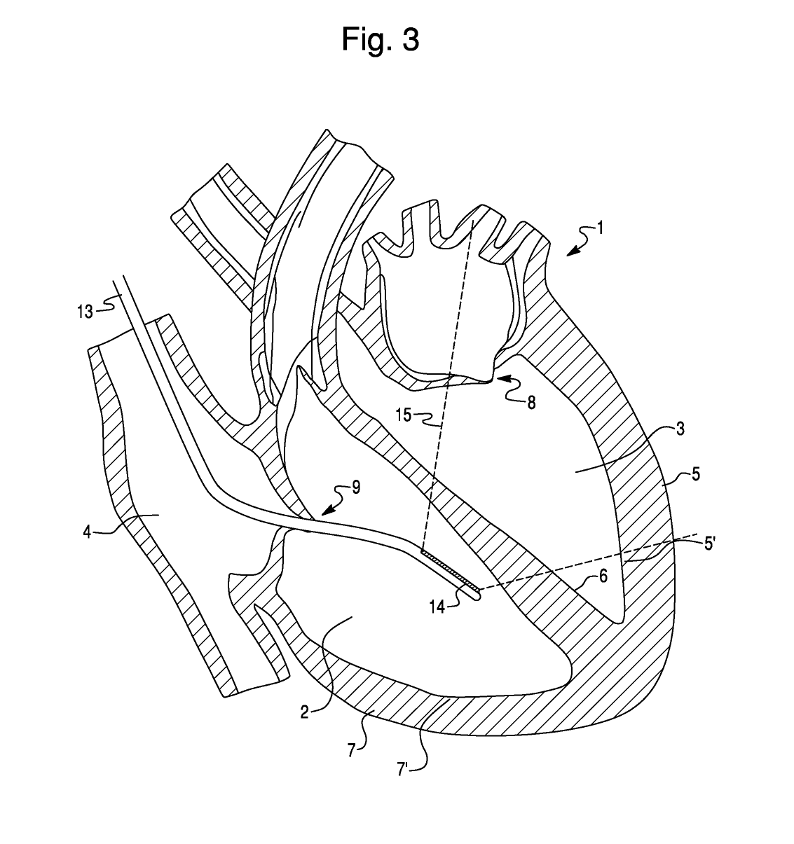

Instead of a using a sheath 10 to position the ultrasound phased array catheter 13 in the heart 1, a steerable ultrasound catheter, such as disclosed in U.S. Patent Publication 2005/0228290, can be used and guided directly under fluoroscopy control into position within orifice of the tricuspid valve or within the right atrium, as illustrated in FIG. 3. FIG. 3 illustrates positioning of the ultrasound sensor 14 in the right ventricle 2 with catheterization via the subclavian or jugular veins.

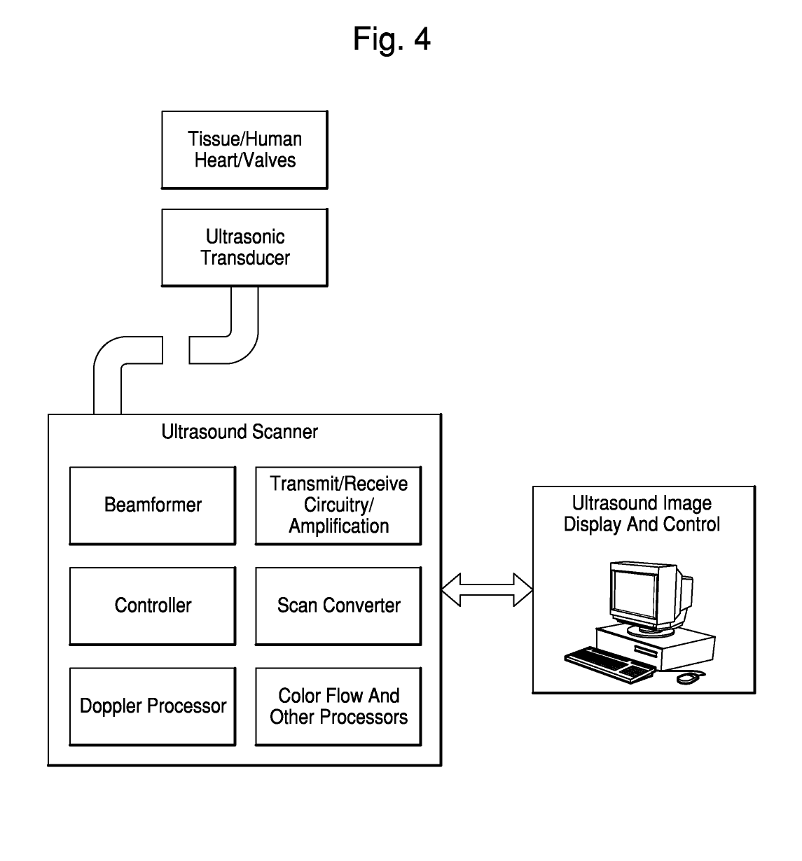

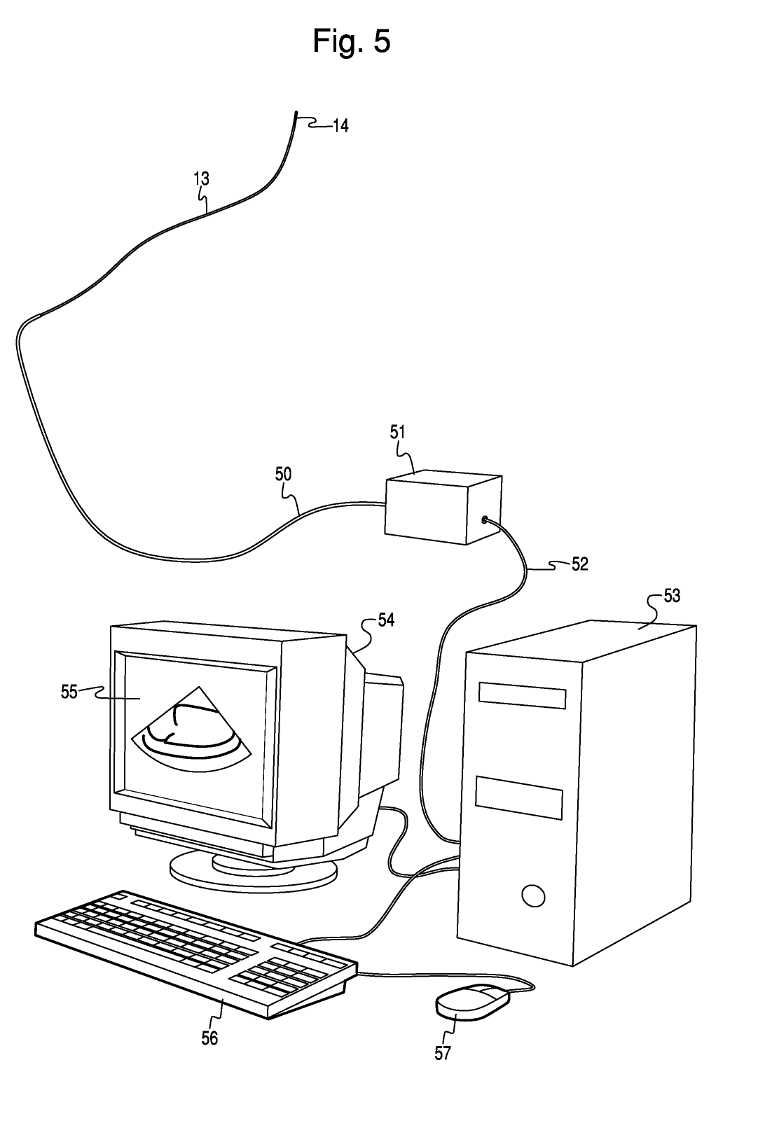

With the catheter phased array transducer 14 properly positioned within the heart, an ultrasound system, such as the ViewMate® Intracardiac Ultrasound Catheter System manufactured by EP MedSystems, Inc. of West Berlin, N.J., is connected to the catheter, an example of which is illustrated in FIGS. 4 and 5. The ultrasound system generates the electrical pulses which cause the transducer elements to emit ultrasound pulses. The ultrasound system also receives and processes the resulting echoes detected by the transducers. An ultrasound system includes a data cable 50 connected between the catheter 13 and an electrical isolation box 51. The data cable 50 may be connected to a handle (not shown) on the catheter 13 or may be an extension of the catheter itself. A data cable typically includes a number of coaxial cables, one for each phased array transducer element. The electrical isolation box 51 electrically isolates the catheter, thereby protecting the patient from stray currents that may be induced in the system or cabling 52 by radio frequency emissions and from fault currents that may result from an electrical short within the system equipment. An example of a suitable electrical isolation box 51 is described in U.S. patent application Ser. Nos. 10/997,898, published as U.S. Publication No. 2005-0124898 to Borovsky et al., and 10/998,039, published as U.S. Publication No. 2005-0124899 to Byrd et al., the entire contents of both of which are incorporated herein by reference in their entirety. Connected to the electrical isolation box 51, maybe another data cable 52 which conducts electrical information to a system processor 53. Coupled to the system processor 53 will typically be a monitor 54 for presenting a display 55 of the ultrasound data, and a keyboard 56 and pointing device 57 and/or other human interface device for accepting user commands and data inputs.

When the catheter is positioned within a patient's heart, the ultrasound system generates electrical pulses which cause the ultrasound transducers in the phased array transducer 14 to emit ultrasound pulses. By a controlling the phase lag of the pulses emitted by each transducer element within the phased array, a combined sound wave is generated with a preferential direction of propagation. Echoes from structures within the heart are received by the transducer elements and transformed into electrical pulses by the transducer. The electrical pulses are carried via the cables 50, 52 to the processor 53. The processor 53 analyzes the electrical pulses to calculate the distance and direction from which echoes were received based upon the time of arrival of the echoes received on each transducer element. In this manner, ultrasound energy can be directed in particular directions, such as scanned through a field of regard 15, and the resulting echoes interpreted to determine the direction and distance from the phased array that each echo represents.

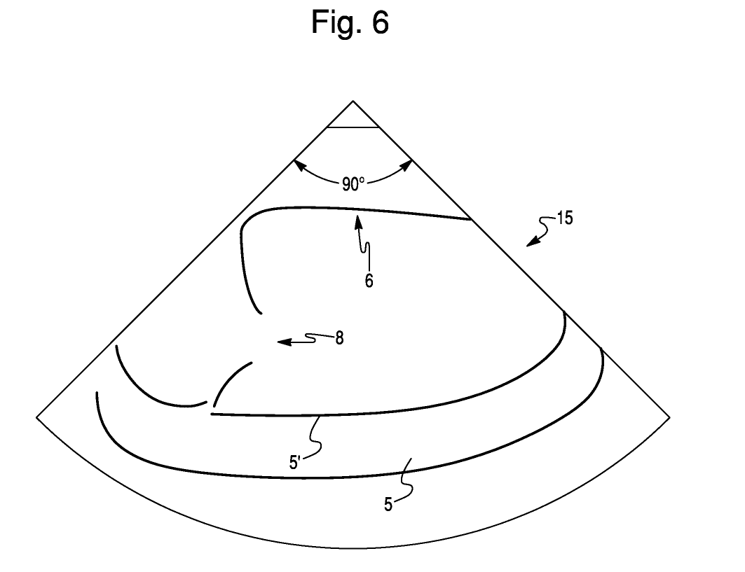

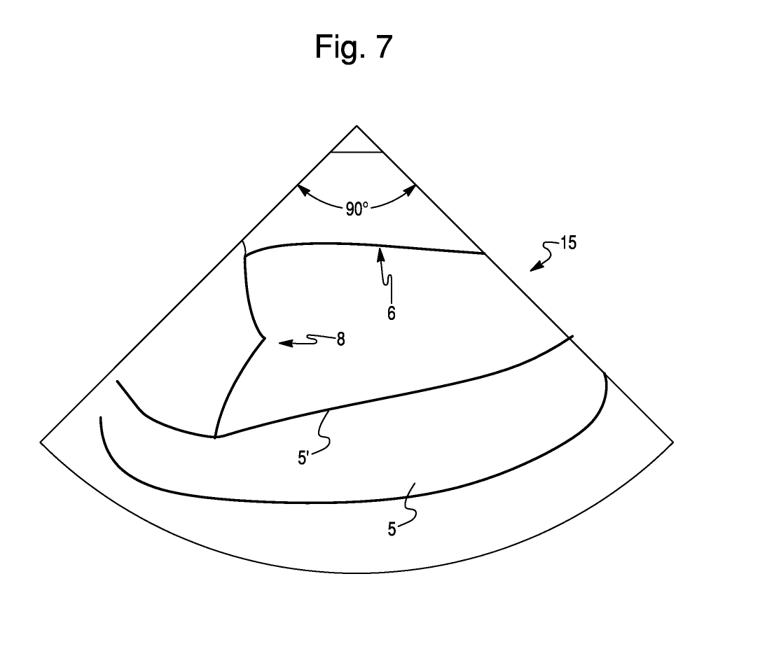

Scanning the ultrasound energy through a field of regard 15 generates a two-dimensional (2D) image of the heart, examples of which are shown in FIGS. 6 and 7. After a 2D scan is obtained, the catheter phased array transducer is rotated and another 2D image obtained, so that most of the endocardial surface of the ventricle (left or right) can be imaged. The B-mode ultrasound imaging technique is employed in this process. B-Mode ultrasound imaging displays an image representative of the relative echo strength received at the transducer. A 2-D image can be formed by processing and displaying the pulse-echo data acquired for each individual scan line across the angle of regard 15 of the phased array transducer. This process yields a two-dimensional B-mode image of the endocardial surface of the ventricle, examples of which is illustrated in FIGS. 6 and 7. Such images are obtained and recorded during approximately 10 or more cardiac cycles.

Since the scan rate of a phased array ultrasound transducer is much faster than the cardiac cycle, each scan presents a 2-D image at a particular time or phase in the cycle. Thus, individual scans, or a plurality of scans obtained at a particular phase or relative time within the cardiac cycle over a number of beats combined into an average image, can be used to provide a “freeze frame” image of the heart at particular instants within the cardiac cycle. Methods for combining and averaging multiple scans at a particular phase or relative time within the cardiac cycle (time gating) are described in U.S. application Ser. No. 11/002,661 published as U.S. Patent Publication No. 2005/0080336 to Byrd, et al., the entire contents of which are incorporated herein by reference in their entirety.

The freeze frame capability of B-mode images is used to obtain recordings particularly at the onset of QRS complex, which is near the end of diastole, and at the beginning of the T wave which is near the end of systole. FIG. 6 illustrates B-mode image of the left ventricle at diastole, and FIG. 7 illustrates a B-mode image of the left ventricle at systole. Sensing the QRS complex and T wave measurements obtained by electrocardiogram (ECG) sensors provides a signal that can be used to select a particular single image, or collect a number of images for averaging at the points of diastole and systole. The ECG sensors may be placed intracardiac via an electrode catheter or on the chest.

Automated edge-seeking algorithms or manual delineation of the endocardial signals is performed on the obtained images throughout the entire ventricle. Edge-seeking algorithms locate the edges of structure (e.g., ventricle walls) by noting a steep change in brightness (indicating echo intensity) from pixel to pixel. Alternatively, the cardiologist may define the edge of the endocardial surface 5′, 7′ in the image by manually tracing the edge using an interactive cursor (such as a trackball, light pen, mouse, or the like) as may be provided by the ultrasound imaging system. By identifying the edges of structure within an ultrasound image, an accurate outline of ventricle walls can be obtained and other image data ignored. The result of this analysis is a set of images and dimensional measurements defining the position of the ventricle walls at the particular instants within the cardiac cycle at which the “freeze frame” images were obtained. The dimensional measurements defining the interior surface 5′ or 7′ of the endocardium can be stored in memory of the ultrasound system and analyzed using geometric algorithms to determine the volume of the ventricle.

For the left ventricle 3, an image of most of if not the entire endocardium is obtained, preferably from the base of the aortic valve to the left ventricular apex and across back to the base of the aortic valve. An illustration of such an ultrasound image at diastole is provided in FIG. 6. The aortic valve plane is imaged and defined using edge-seeking algorithms to complete the delineation of the cavity enclosing the blood flow. In particular, these images are obtained for the end-diastolic and end-systolic portions of the cardiac cycle, FIGS. 6, 7, thereby measuring the dimensions and contours of the ventricle walls at the instances of maximum (FIG. 6) and minimum volume (FIG. 7).

Having obtained dimensional measurements of the left ventricle 3 from the ultrasound images at or near diastole and systole, the ultrasound system processor can calculate the volume in the ventricle at both instances and, from the ratio of these two volumes, calculate the ejection ratio of the left ventricle 3.

While FIGS. 6 and 7 and the foregoing description address the left ventricle 3, similar images are obtained for the right ventricle 2, except that the image extends from the base of the tricuspid value 9 to the right ventricular apex 93 and across back to the base of the tricuspid value 9. From the images of the right ventricle 2, similar calculations of ventricle volume are obtained at points in the cardiac cycle of maximum and minimum volume to calculate the ejection fraction of the right ventricle 2.

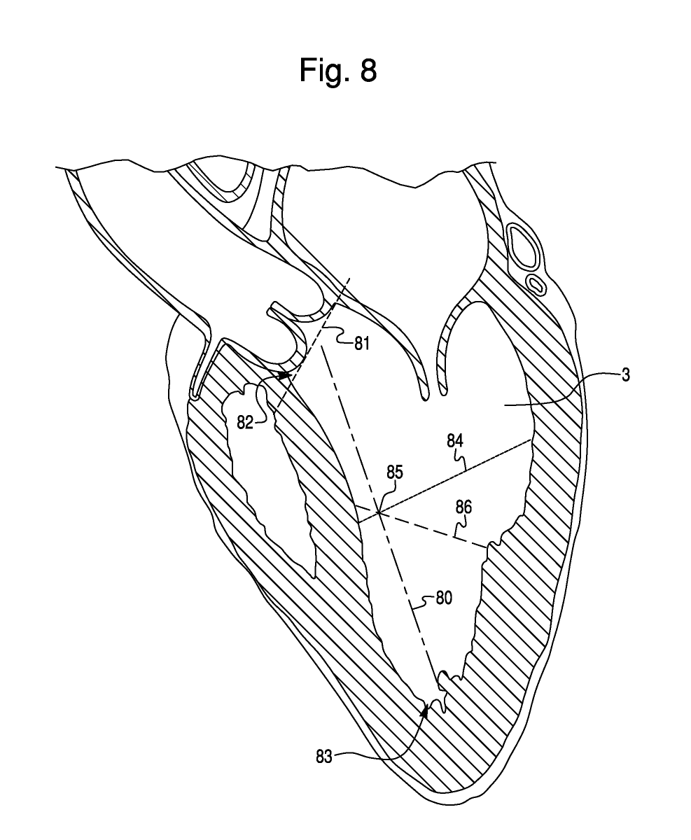

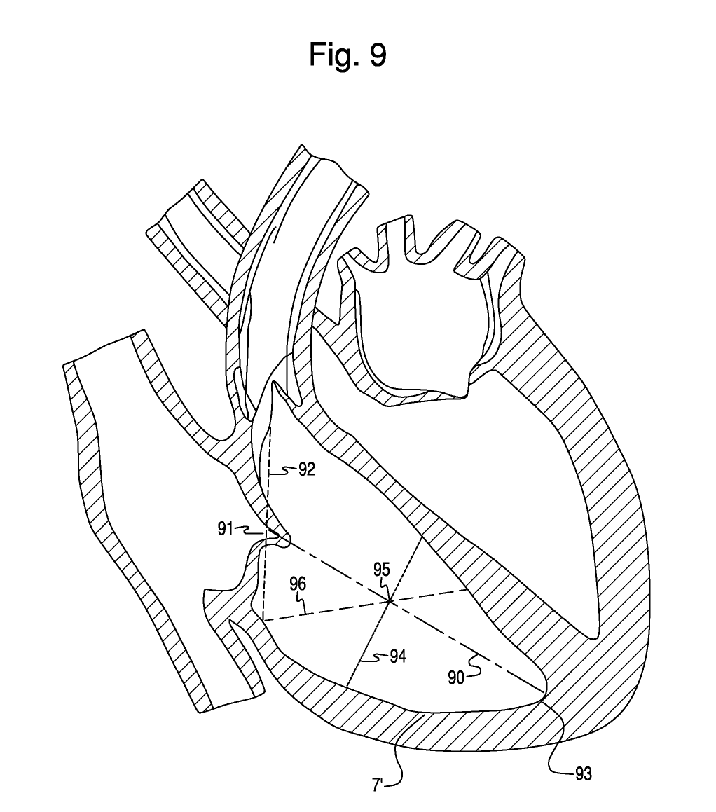

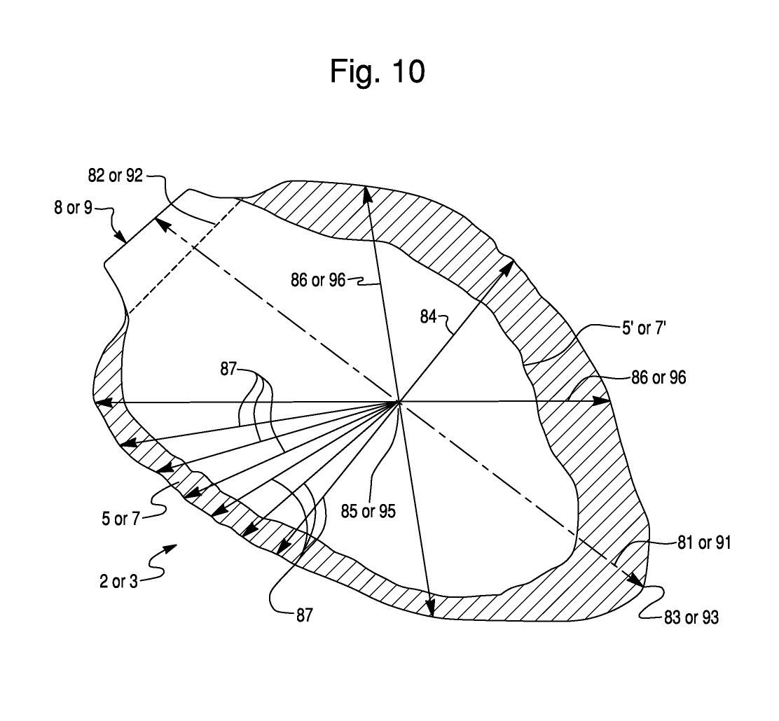

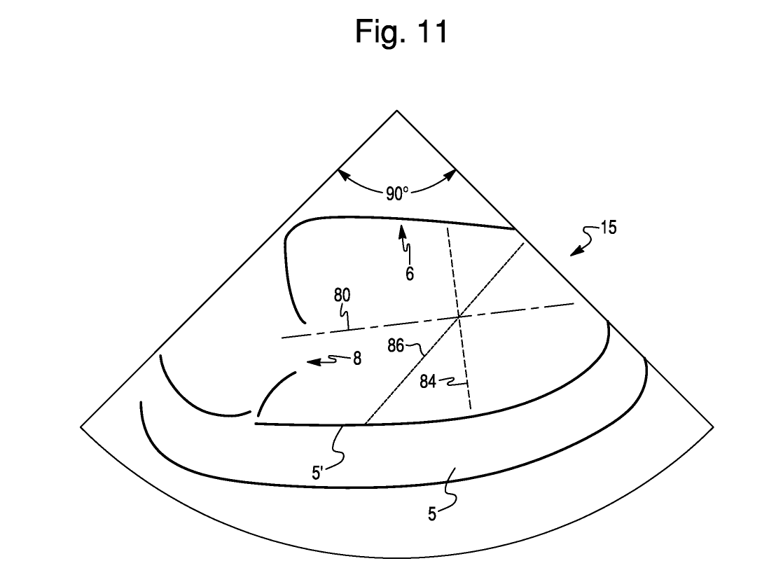

Another embodiment of the invention provides a method for the detecting and analyzing the segmental, regional, and global pumping efficiencies of the ventricles. In this embodiment, the long axis 80 of the left ventricle 3 is defined from the mid point 81 of the aortic valve plane 82 to the left ventricular apex 83, as illustrated in FIG. 8. Similarly, the long axis 90 of the right ventricle 2 is defined from the mid plane 91 of the tricuspid of the pulmonic valve plane 92 to the right ventricular apex 93. The long axis 80, 90 from the midpoint of the valvular plane to the apex is then subtended and bisected. The perpendicular axis 84, 94 at the midpoint 85, 95 of the long axis 80, 90 is used for subtending the short axis at a perpendicular. Additional radians 86, 96 are then subtended at an acute angle, such as 30 or 45 degree angles, from the central point 85, 95 of the ventricle as defined by the intersection of the two axes. These radial axes are superimposed along with the short and long axes on the end-systolic and end-diastolic frames of the ventricle B-mode image, as illustrated in FIG. 10 for the left ventricle.

The area in each segment as defined by the radial axes is then planimetered and automatically computed. The area in each sector of the ventricle or the fractional shortening along the radian in the sector can be used as a measure of regional ventricular function and ejection fraction. The difference in area between the measured area in the end-diastolic image and the measured area in the end-systolic image characterizes the regional ejection fraction for the region of the heart subtended by each such pair of corresponding sectors and may be used to estimate the regional ejection fraction for the measured segment. This estimate is based upon the assumption that the length of the long axis 80, 90 does not change significantly during contraction, so that the change in volume is proportional to the change in area of a transverse cross section. In this manner, the regional ejection fraction for each of the segments can be easily calculated by the ultrasound system processor to provide ejection fractions for multiple regions of the ventricle.

The definition of axes and radians is further illustrated in FIG. 10 which shows a stylized ventricle which may be either the left ventricle 3 or right ventricle 2. Referring to FIG. 10, an embodiment method defines a long axis 90 to extend from the midplane of the tricuspid 9 of the pulmonic valve plane to the right ventricular apex 93. For the left ventricular cavity 3, the method defines the long axis 80 to extend from the mid point 91 of the aortic valve plane 91 to the left ventricular apex 83. The long axis 80, 90 from the midpoint 81, 91 of the valvular plane 82, 92 to the apex 83, 93 contains a midpoint 85, 95, which bisects the long axis 80, 90. A transverse line or plane 84, 94 is defined at the midpoint perpendicular to the long axis 80, 90. Radials 86, 96 are then defined in the plane of the cross-sectional image at an acute angle to the transverse axis 84, 94 and crossing the midpoint 81, 91. The embodiment may construct further radials 87 extending from the midpoint 85, 95 of the long axis 80, 90 at a plurality of angles (e.g., multiples of 30 or 45 degrees) with respect to the long axis 80, 90. Each radial 87 terminates where it intersects the endocardial wall 5′ or 7′ in the ultrasound image. Each half of the long axis 80, 90 also forms a radial.

The embodiment method may approximate the area of each sector or region in an image of the ventricular cavity 2 or 3 being examined as the sum of the areas of multiple, small, disjoint, abutting triangles which effectively subdivide and cover the sector or region. For example, each triangle may have the long axis bisection point 85, 95 as one vertex, and two sides defined by radials 87 from the bisecting midpoint 85, 95 terminating at the edge of the endocardial wall 5′ or 7′.

As an alternative or addition to the area method of estimating ejection fraction, the change in length of each of the radials 84, 86, 87 can provide information characterizing the instantaneous ejection fraction by monitoring the endocardial wall motion in the direction along each radial. These radials 84, 86, 87 relate to specific anatomic regions of the imaged heart ventricle. The values and relative timing of the regional ejection fractions, which correspond to the various radials 84, 86, 87, can be used to assess the effect of alternative interventions as described herein.

This embodiment for calculating regional ejection fractions can also be accomplished at various predefined points in the systolic cycle, such as, for example, at or near early (˜33%), mid (˜50%), late (˜67%) and end (˜100%) points of the systolic period of ventricular contraction. This can be accomplished by subtracting the area of each segment at the predefined point in the cycle from the area of the segment measured at diastole. This evaluation provides a novel and useful method for detecting and diagnosing ventricular dysynchronous contraction.

In another embodiment of the invention calculates an overall global ejection fraction by summing all of the regional ejection fractions obtained according to the above method. The global ejection fraction can be measured at different predefined points in the systolic cycle, such as at or near early (˜33%), mid (˜50%), late (˜67%) and end (˜100%) points of the systolic period of ventricular contraction. This calculation permits evaluation of ventricular ejection fraction at different points in the cardiac cycle. By calculating the ventricular ejection fraction at different points in the cardiac cycle, detection of ventricular dysynchronous contraction is possible.

In another embodiment, instead of defining a long axis 80, 90 of the ventricle, the ultrasound processor can compute the centroid of the edge trace of the endocardium wall and bisect the edge trace about the centroid to define a point from which to extend radians for calculating ejection fraction according to the methods described herein.

Under normal circumstances, the overall ejection fraction increases during systole until the end of systole. The various embodiment methods allow for estimation of ejection fraction when segments of the ventricle are not contracting in coordination with one another. In ventricular dysynchronous contraction, some portions of the ventricle wall contract out of phase (early, late or not at all) with the rest of the ventricle. Such ventricular dysynchronous contraction results in ineffective or incomplete ejection of blood from the heart, which is indicative of heart disease and can lead to formation of blood clots which may cause embolisms or stroke. This embodiments enable estimation and detailed analysis of the ejection fraction as it changes over the course of a cardiac cycle, especially when regions of the ventricle are not contracting in normal coordination.

In an embodiment, regional ejection fraction during the systolic period can be monitored in a phased analysis of the regional wall motion to give the temporal sequence of regional ventricular ejection. The phase analysis determines the timing of wall motion each of the ventricular segments with respect to each other.

In another embodiment, an axial assessment of ventricular function can be performed along the long axis of the ventricle using methods similar to those for measuring along radians to determine apical-to-basal sequencing of ventricular ejection.

In another embodiment, fractional shortening along each of the radial axes is measured to allow for assessment of instantaneous fractional shortening. This embodiment is as an alternative to the area method for computation of regional wall motion embodiment method described above. In this embodiment, the regions defined by the radial axes can be related to specific anatomic regions of the heart ventricle to assess the effect of interventions as described herein. By measuring the shortening of each radial axis defined within the ventricle, a simple measure of the phase and relative contraction of regions of the ventricle is obtained. In a situation where the clinician seeks to identify regions of a ventricle that are lagging during contraction, such as in a condition of incoordinate ventricle contraction, a simple measure of radian length versus time is sufficient to identify the timing and relative magnitude of regional contraction motions.

In the various embodiments, the ultrasound system processor will complete the analysis by generating a display of the computed regional and global ejection fractions, or regional fractional shortening (contraction) measurement, in some useful format for inspection by the cardiologist. The format of the display may simply be the regional ejection fractions presented as numbers related to their respective sectors. Each number can be superimposed and centered on its corresponding sector on the image taken at the time of the cycle for which the set of ejection fractions were computed. Alternatively, the numbers can be superimposed on a stylized model, cartoon or image (e.g., an X-ray image) of the heart. Similarly, where the measured factor is relative timing of contraction movements of different regions of the ventricle, as may be measured to detect, diagnose and/or treat ventricle dyssynchrony or incoordinate ventricular contraction, the relative contraction time or delay can be displayed superimposed on the corresponding sector

Superimposing measured performance values on an image or stylize model of the heart can reveal the performance of each ventricle sector or region at the time in the cardiac cycle corresponding to the particular ultrasound image. Such a display can aid the clinician in identifying ventricle regions that have poor function or exhibit lagging or inadequate movement during the contraction cycle. Use of a stylized model of the heart may aid the clinician in recognizing the particular regions of the ventricle involved, particularly since ultrasound images sometimes include speckle and other noise which may render the image difficult to understand. Thus, by presenting the ventricle performance measures superimposed upon a heart image or model, the various embodiments assist the clinician in identify locations of dysfunction and, in particular, sites for administering intervention or therapy, such as sites for attaching pacemaker pacing leads to the ventricle wall.

In another embodiment of the display function, the system processor may perform a computational step to plot or generate a line graph representing the value of the regional ejection fraction, contraction movement or radian length for a given sector or radian of the ventricle as it changes over the cardiac cycle. In such a graph, one axis can represent the sequential phases of the cardiac cycle, and another axis represents the ejection fraction (or contraction movement or radian length) for the sector over the time of a cardiac cycle. Plots of the various ventricle sectors may be superimposed (or plotted one over the other) in a display to show the timing relationship of contraction of the sectors. The plotted line of each sector may be represented by a different graph line in the display, where each line is distinguished by color or style (solid, dashed, dotted, and so forth). In this manner, the plots can further reveal the relative temporal motion of the regions of the heart to which the sectors correspond. Such a display will graphically reveal ventricle regions that lag or contract in an uncoordinated fashion relative to the rest of the ventricle and provide an easy to interpret summary of the relative synchronization of various regions of the ventricle. This display can also be used to identify a region or regions that will benefit from pacing, and thus aid in identifying optimum locations for positioning pacing leads within the ventricle.

Data for a formatted display may be computed from a single cardiac cycle or systolic portion of a single cycle. Alternatively, data in the formatted display (whether numeric or graphical) for a given sector and a given time in the cardiac cycle can be the average of many measurements for the given sector at the same given time in each cycle of a plurality of cardiac cycles (for example, ten consecutive cycles).

In another embodiment of the display function, the ultrasound images may be ultrasound system processor can monochromatically shade each ventricle sector or region using a color representing the value of the local quantitative assessment measurement (e.g., ejection fraction, contraction wall movement and regional radian length) computed for the time the image was acquired. For example, red may correspond to the greatest ejection fraction, blue corresponds to the least ejection fraction, and other colors of the spectrum between red and violet (orange, yellow, green, and cyan) correspond to intermediate values. Such color coding can efficiently communicate the measured heart function parameter without masking the image or stylized model of the heart, and thus aid the cardiologist in identifying locations for intervention or treatment.

In a variation of the preceding embodiment, the display can use color to indicate relative timing (i.e., phase within the cardiac cycle) at which the quantitative assessment measurements (e.g., ejection fraction, contraction wall movement and regional radian length) of each ventricle sector or region peaks in the cardiac cycle (i.e., when in the cardiac cycle the contraction ceases for each region). For example, red may be used to shade those regions in which the ejection fraction value peaks first in the cardiac cycle, with other colors of the spectrum (orange, yellow, green, blue, violet) shading the regions for which the local ejection fraction value peaks at relatively later points in the cardiac cycle.

In yet another embodiment, the ultrasound system processor can be programmed to display a replay of the acquired ultrasound images and associated quantitative assessment measurements (e.g., ejection fraction, contraction wall movement and regional radian length) in slow motion or stepwise under cardiologist control.

The various embodiments are intended to aid the cardiologist in recognizing, diagnosing and treating various ventricular function maladies. By measuring and displaying ejection fraction, both globally and regionally, the cardiologist can identify the need for treatment. By displaying the quantitative assessment measurements (e.g., ejection fraction, contraction wall movement and regional radian length) against an image or stylized model of the ventricle, the various embodiments enable the cardiologist to identify particular locations in the ventricle for intervention and treatment.

Examples of interventions and treatments that may be assessed using the various embodiment method include, for example, restoration of blood flow (angioplasty), insertion of a stent, resynchronization of ventricular contraction by use of implantable heart devices (e.g., biventricular and multi-ventricular pacing techniques), the use of drugs to study the performance of these heart segments, and combinations of these treatments. In particular, the embodiments which provide regional quantitative assessment measurements (e.g., ejection fraction, contraction wall movement and regional radian length) graphically localized on a heart image or model at different phases of the cardiac cycle can reveal to the cardiologist regions of the ventricle which require pacing in order to resynchronize or re-coordinate ventricle contraction. For example, a region that contracts late—and thus out of phase—with surrounding portions of the ventricle, may be a suitable site for attaching a pacemaker pacing lead. In this manner, the pacing lead can be positioned at the region which will most benefit from the pacing stimulus. Displaying the results on an image or model of the heart may also help the cardiologist plan and implement the procedure for attaching the pacer lead in the selected region.

The various embodiments can also be employed to quantitatively assess the impact of the intervention(s) by being repeated after the intervention. In this embodiment, the measurements and calculations according to the embodiment methods described herein are obtained before and after the intervention and the results compared. In this manner, measurements taken before the intervention provide a baseline quantitative assessment measurement (e.g., ejection fraction, contraction wall movement and regional radian length) which can be taken to measurements obtained after the intervention. Comparisons may be on a global or regional basis to assess effects of the intervention on both total ejection fraction and on regional ejection fraction and ventricular dysynchronous contraction.

This embodiment provides the cardiologist with quantitative assessments of the impact of the treatment which can be used to modify or adjust subsequent interventions. In particular, this embodiment can assist the cardiologist in selecting pacemaker settings, such as timing and pulse wave forms (e.g., magnitude, pulse width, and pulse phase), which yield optimized ventricular function. In this manner, the measurements and calculations according to the embodiment methods described herein are obtained after a particular set of pacemaker settings are implemented. The pacemaker settings are then adjusted and the measurements repeated. If the quantitative assessment measurement improves, the cardiologist may continue adjusting the parameter (such as pace timing) until subsequent measurements indicate degraded ventricular function. If the quantitative assessment measurement degrades after a setting change, the cardiologist may reverse the direction of parameter adjustment (e.g., increasing or decreasing the pacing lag time) and repeat the quantitative assessment measurement. In this manner, the cardiologist can use the quantitative assessment measurements according to the various embodiments in order to optimize pacemaker based on measured ventricular function.

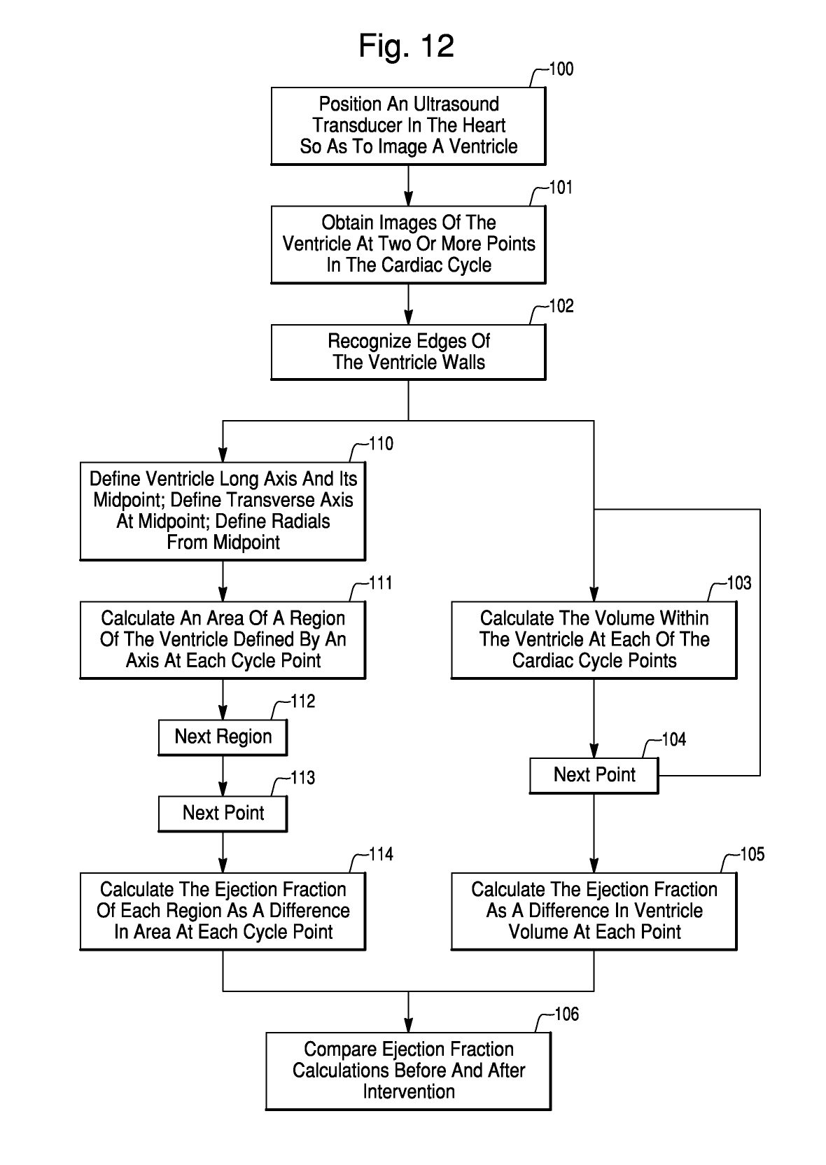

FIG. 12 provides a flow process diagram of the various embodiment methods described above. Referring to FIG. 12, as a first step 100, the physician positions an ultrasound catheter in the heart so the ultrasound transducer can image either the left or right ventricle. In step 101, ultrasound images are obtained at two or more points in the cardiac cycle as described herein. In step 102, edge recognition algorithms or manual edge tracing techniques are used to recognize the edges of the ventricle walls. According to one embodiment, measurements of distances between ventricle walls are used to calculate the volume within the ventricle at one of the points in the cardiac cycle, step 103. This calculation is performed for each point in the cardiac cycle for which images were obtained, loop 104. In step 105, the ejection fraction is determined as the difference in ventricle volume at each of the two or more points in the cardiac cycle. Finally, the method can be performed before and after an intervention to compare the ejection fractions to determine if there was any impact from the intervention, step 106.

In another embodiment, after the edges of the ventricle walls have been recognized, step 102, the method constructs a number of axis and radians for measuring ventricle ejection fraction, step 110. Step 110 can be performed for either ventricle 2 or 3. For the right ventricular cavity 2, the algorithm of step 110 defines a long axis 90 to extend from the mid plane of the tricuspid 9 of the pulmonic valve plane to the right ventricular apex 93. For the left ventricular cavity 3, the algorithm of step 110 defines the long axis 80 to extend from the mid point 91 of the aortic valve plane 91 to the left ventricular apex 83. The algorithm of step 110 defines a midpoint 85, 95, which bisects the long axis 80, 90 at the midpoint between the valvular plane 82, 92 and the apex 83, 93. The algorithm of step 110 then constructs a transverse line or plane 84, 94. The algorithm of step 110 then constructs radials 86, 96 in the plane of the cross-sectional image at an acute angle to the transverse axis 84, 94 crossing the midpoint 81, 91. The algorithm of step 110 may construct further radials 87 extending from the midpoint 85, 95 of the long axis 80, 90 at a plurality of angles (e.g., multiples of 30 or 45 degrees) with respect to the long axis 80, 90. Each radial 87 terminates where it intersects the endocardial wall 5′ or 7′ in the ultrasound image. Each half of the long axis 80, 90 also forms a radial. The area of each region defined by the axes and radians is the calculated, step 111. The area calculation in step 111 is repeated for each region, loop 112, and for each point in the cardiac cycle at which images were obtained, loop 113. The ejection fraction of each region is then calculated as the difference in area in the region between two points in the cardiac cycle, step 114. Step 114 may be repeated for each region to obtain all regional ejection fractions. Finally, the method can be performed before and after an intervention to compare the ejection fractions to determine if there was any impact from the intervention, step 106.

While the foregoing description and FIG. 12 depict the method steps as occurring in a particular order, such order is for example purposes only and the steps may be accomplished in a different sequence or in combination with additional steps without departing from the scope and spirit of the present invention.

While the present invention has been disclosed with reference to certain preferred embodiments, numerous modifications, alterations, and changes to the described embodiments are possible without departing from the sphere and scope of the present invention, as defined in the appended claims. Accordingly, it is intended that the present invention not be limited to the described embodiments, but that it have the full scope defined by the language of the following claims, and equivalents thereof.

What is claimed is:

1. A method for evaluating the pumping function of a heart, comprising:
positioning an ultrasonic catheter in the heart so that a phased array transducer located on the ultrasonic catheter can image an endocardial surface of a ventricle;
generating an image of the endocardial surface at two or more points in a cardiac cycle;
measuring a dimension of the endocardial surface at the two or more points in the cardiac cycle; and
calculating an ejection fraction based upon the measured dimensions at the two or more points in the cardiac cycle.

2. The method according to claim 1, wherein generating an image of the endocardial surface comprises generating an image from a base of an aortic valve to the ventricular apex and across the base of the aortic valve.

3. The method according to claim 2, wherein generating an image of the endocardial surface is performed at a plurality of points over an entire cardiac cycle from end-diastolic to end-systolic.

4. The method according to claim 1, wherein the two or more points in the cardiac cycle are near diastole and near systole.

5. The method according to claim 1, wherein the two or more points in the cardiac cycle are at or near early (˜33%), mid (˜50%), late (˜67%) and end (˜100%) points of the systolic period of ventricular contraction.

6. The method according to claim 1, wherein positioning an ultrasonic catheter in the heart comprises positioning the ultrasound catheter over a tricuspid valve of the right atrium oriented so as to obtain an ultrasound image of the right ventricle.

7. The method according to claim 1, wherein positioning an ultrasonic catheter in the heart comprises positioning the ultrasound catheter within the right ventricle oriented so as to obtain an ultrasound image of the left ventricle.

8. The method according to claim 6, further comprising positioning an ultrasonic catheter within the right ventricle oriented so as to obtain an ultrasound image of the left ventricle.

9. The method according to claim 1, wherein measuring a dimension of the endocardial surface comprises measuring an area defined by an axis of the ventricle.

10. The method according to claim 9, wherein calculating the ejection ratio comprises calculating a difference in area defined by the axis at a first point in the cardiac cycle and at a second point in the cardiac cycle.

11. The method according to claim 9, wherein the axis is defined as a line perpendicular to and positioned at a midpoint of a long axis of the ventricle, wherein the long axis of the ventricle is defined from the mid point of a valve plane to the ventricular apex.

12. The method according to claim 1, wherein:
measuring a dimension comprises measuring an area defined by a first line perpendicular to and positioned at a midpoint of a long axis of the ventricle and a second line subtended at an acute angle from the long axis and positioned at the midpoint of the long axis;
the long axis ventricle is defined from the mid point of a valve plane to the ventricular apex; and
the ejection ratio is a regional ejection fraction calculated as a difference in the area at a first point in the cardiac cycle and at a second point in the cardiac cycle.

13. The method according to claim 12, further comprising calculating a global ejection fraction as a sum of all regional ejection fractions.

14. The method according to claim 1, further comprising:
displaying on a visual representation of the heart a change in the measured dimension between the two or more points in the cardiac cycle; and
determining a location for an intervention based upon the display.

15. The method according to claim 14, wherein:
the intervention includes emplacement of a pacemaker; and
determining the location for the intervention comprises identifying a location for attaching a pacing lead to the heart.

16. The method according to claim 1, further comprising:
performing an intervention;
repeating the method of claim 1 after the intervention; and
comparing calculated regional ejection fractions before and after the intervention.

17. The method according to claim 16, wherein the intervention includes emplacement of a pacemaker, further comprising:
adjusting a parameter of the pacemaker;
repeating the method of claim 1; and
comparing calculated regional ejection fractions before and after the adjustment.

18. The method according to claim 12, further comprising:
performing an intervention;
repeating the method of claim 12 after the intervention; and
comparing calculated regional ejection fractions before and after the intervention.

19. The method according to claim 18, wherein the intervention includes emplacement of a pacemaker, further comprising:
adjusting a parameter of the pacemaker;
repeating the method of claim 12; and
comparing calculated regional ejection fractions before and after the adjustment.

20. A method for evaluating regions of a ventricle of a heart, comprising:
identifying an inner endocardial boundary of the ventricular cavity in ultrasound images of the heart acquired at two or more times during the cardiac cycle;
subdividing the images of the ventricular cavity into sectors which subtend regions of the endocardium of the ventricle;
estimating a local ejection fraction for a region using the sectors which subtend the region in the ultrasound images acquired at different times during the cardiac cycle; and
reporting the local ejection fraction of one or more regions.

21. The method according to claim 20, wherein:
a sector is bounded by two radials in the ultrasound image and the image of the inner endocardial boundary;
the radials originate at a midpoint of a line segment between a center of an image of the tricuspid valve and an image of the ventricular apex; and
the radials lie at specified angles with respect to the line.

22. The method according to claim 20, wherein estimating a local ejection fraction for a region comprises computing a change in area of two sectors, wherein:
each of the two sectors is from a different image; and
the two sectors correspond to the same region of the ventricle.

23. The method according to claim 22, wherein the area of a sector is computed by:
subdividing the sector into disjoint triangles approximately subdividing and cumulatively covering the sector; and
summing the areas of all the triangles.

24. The method according to claim 21, wherein estimating the ejection fraction for a region comprises computing a change in length of each radial over a cardiac cycle.

25. The method according to claim 20, further comprising estimating a global ejection fraction as a sum of the local ejection fractions for all the regions.

26. The method according to claim 20, wherein reporting the local ejection fraction of one or more regions comprises displaying a graph line representing the estimated local ejection fraction for a region at various times of the cardiac cycle.

27. The method according to claim 20, wherein reporting the local ejection fraction of one or more regions comprises displaying the images with a color-coded indication of estimated ejection fraction for each sector at the time in the cardiac cycle corresponding to the image.

28. The method according to claim 27, further comprising identifying a location on the heart for an intervention based upon the displayed images and color-coded indications.

29. The method according to claim 28, wherein the intervention includes emplacing a pacemaker and identifying a location comprises identifying a location for attaching a pacing lead to the heart.

30. The method according to claim 29, further comprising:
repeating the steps of claim 20 after a parameter of the pacemaker is adjusted; and
readjusting the pacemaker parameter based upon the reported local ejection fraction.
